(19) United States
(12) Patent Application Publication
(10) Pub. No.: US 2024/0374366 A1
(43) Pub. Date: Nov. 14, 2024

(54) TISSUE SUSPENSION IMPLANT

(71) Applicant: PORIFEROUS, LLC, Newnan, GA (US)

(72) Inventors: Aaron Noble, Newnan, GA (US); Spencer Jones, Newnan, GA (US)

(73) Assignee: PORIFEROUS, LLC, Newnan, GA (US)

(21) Appl. No.: 18/784,442

(22) Filed: Jul. 25, 2024

Related U.S. Application Data
(63) Continuation-in-part of application No. 17/711,174, filed on Apr. 1, 2022.
(60) Provisional application No. 63/170,153, filed on Apr. 2, 2021.

(51) Int. Cl. A61F 2/00; A61B 17/04; A61B 17/06

(52) U.S. Cl. CPC A61F 2/0059; A61B 17/0401; A61B 2017/06176

(57) ABSTRACT

An implant that can function as a tissue suspension implant. Embodiments find particular use in connection with eyebrow or forehead lifts, which require raising of soft tissue and skin of the forehead and brow.

This application is a continuation-in-part of U.S. Non-Provisional application Ser. No. 17/711,174, filed Apr. 1, 2022 titled “Tissue Suspension Implant,” which claims the benefit of U.S. Provisional Application Ser. No. 63/170,153, filed Apr. 2, 2021 titled “Tissue Suspension Implant Device and Method,” the entire contents of all of which are hereby incorporated by reference.

### FIELD OF THE DISCLOSURE

According to certain embodiments of this disclosure, there is provided an implant that can function as a tissue suspension implant or otherwise act as an anchor for a suture or other elongated flexible material. Some embodiments find particular use in connection with eyebrow or forehead lifts, which require raising of soft tissue and skin of the forehead and brow.

### BACKGROUND

Eyebrow or forehead lifts are cosmetic procedures that lift the brows in order to improve the appearance of the forehead, the brow, and the area around the eyes. These procedures raise the soft tissue and skin of the forehead and brow, resulting in a more smooth appearance with fewer wrinkles. Typically, small anchors and sutures are used to secure the tissue in place during these procedures. Improvements to anchors are desirable.

### SUMMARY

Accordingly, the present inventors have designed an anchor for a suture or other elongated flexible material, such as a tissue suspension implant that can receive and secure a suture in place. In one embodiment, there is provided a tissue suspension implant, comprising: an implant head; a shaft extending down from the implant head comprising a through hole; and a positioner pin extending up from the implant head for use in positioning the tissue suspension implant. The implant head may have a domed shape and/or tapered head edges.

The implant may be packaged with a drill bit having a drill bit end that forms an opening in a patient's bone that is a fraction of a millimeter smaller than the diameter of the shaft.

The terms “invention,” “the invention,” “this invention” “the present invention,” “disclosure,” “the disclosure,” and “the present disclosure,” used in this patent are intended to refer broadly to all of the subject matter of this patent and the patent claims below. Statements containing these terms should be understood not to limit the subject matter described herein or to limit the meaning or scope of the patent claims below. Embodiments of the invention covered by this patent are defined by the claims below, not this summary. This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings and each claim.

### BRIEF DESCRIPTION OF THE DRAWINGS

FIG. 1 is a side plan view of one embodiment of a tissue suspension implant described herein.

FIG. 2 is a bottom perspective view of the implant of FIG. 1.

FIG. 3 is a top perspective view of the implant of FIG. 1.

FIG. 4 is a side perspective view of an alternate embodiment of a tissue suspension implant.

FIG. 5A is a side perspective view of the implant of FIG. 4 with a protrusion around the through hole for compression after insertion.

FIG. 5B is a side plan view of the implant of FIG. 5A.

FIG. 6 is a side perspective view of an alternate embodiment of a tissue suspension implant, having an oblong and lowered through hole.

FIG. 7 is an exploded view of an alternate embodiment of a tissue suspension implant, having side legs.

FIG. 8 is a perspective view of the implant of FIG. 7.

FIG. 9 is a side plan view of one embodiment of a drill bit that may be used to prepare an opening in bone to receive one of the tissue suspension implants described herein.

FIG. 10 is a perspective view showing an opening in a patient's bone.

FIG. 11 is a perspective view of the patient of FIG. 10 with one of the tissue suspension implants described herein positioned within the opening.

### DETAILED DESCRIPTION

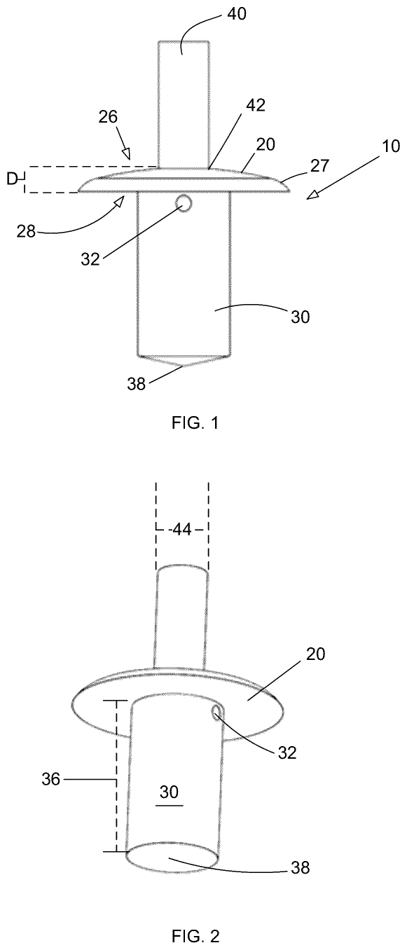

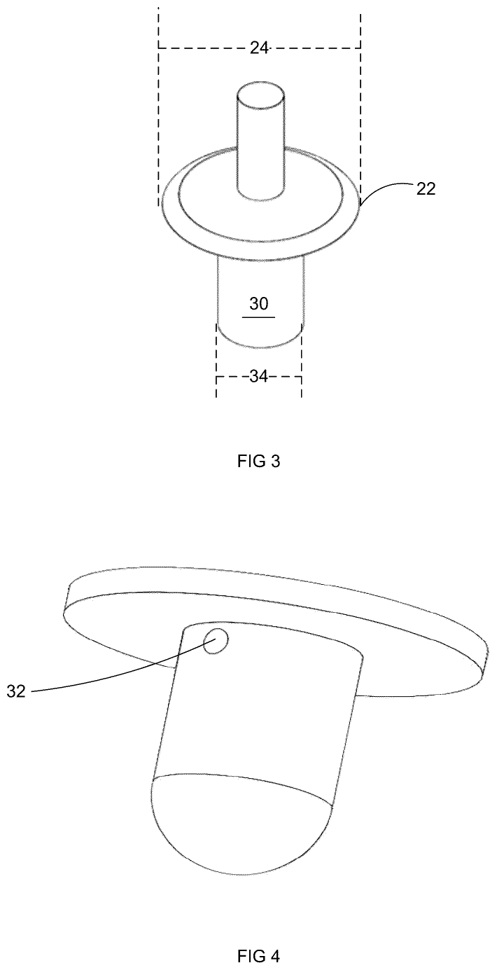

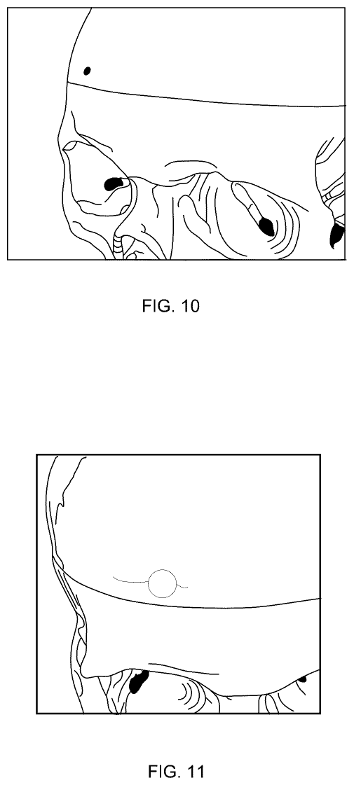

The device described herein is useful for use as a tissue suspension implant 10 or other type of bone anchor. The tissue suspension implant 10 is designed to be received by an opening that is made in a patient's bone, as shown by FIGS. 10 and 11. As shown by FIGS. 1-8, the implant 10 may be shaped somewhat like a pushpin, with a circular implant head 20 and an extending shaft 30. The shaft 30 of the implant is received by the opening in bone. The shaft 30 can be designed to receive and secure a suture. In the examples shown by FIGS. 1-6, the shaft 30 is provided with a through hole 32 that extends laterally through the shaft 30. The suture may be resorbable or non-resorbable, and up to any diameter that can be through the through hole 32 of the implant. The through hole 32 may be formed as a circular hole, as shown by FIGS. 1-5. Alternatively, the through hole 32 may be formed as a slotted tunnel or oblong channel, as shown by FIG. 6. It should be understood that the through hole 32 may be any other appropriately-shaped and sized through hole. It is possible for an implant offering to be provided with multiple different types of shaped suture holes. For example, various options of these holes/tunnels/channels/slots are shown by the figures herein. The general goal is that the through hole 32 is shaped and sized appropriately to receive and allow a needle and an accompanying suture material to extend through the shaft.

In a specific example, the through hole 32 is positioned immediately below the implant head 20. The through hole 32 may be positioned close to the implant head 20 as shown by FIGS. 1-5. In an alternate embodiment, the through hole 32 may be positioned further down along the implant shaft 30, as shown by FIG. 6. Although circular through holes are shown as closer to the implant head 20, it should be understood that an oblong through hole (e.g., of the type shown by FIG. 6) may be positioned closer to the implant head. Similarly, although the oblong through hole is shown as positioned further down along the implant shaft, it should be understood that a circular through hole may be positioned there instead. Various options and different configurations are possible. Additionally, multiple varied implants 10 can be provided in one implant kit in order to provide a surgeon with varied options.

In a specific example, the through hole 32 is circular and the diameter of the through hole 32 may be between about 0.25-0.75 mm. In a more specific example, the diameter of the through hole 32 is about 0.50 mm.

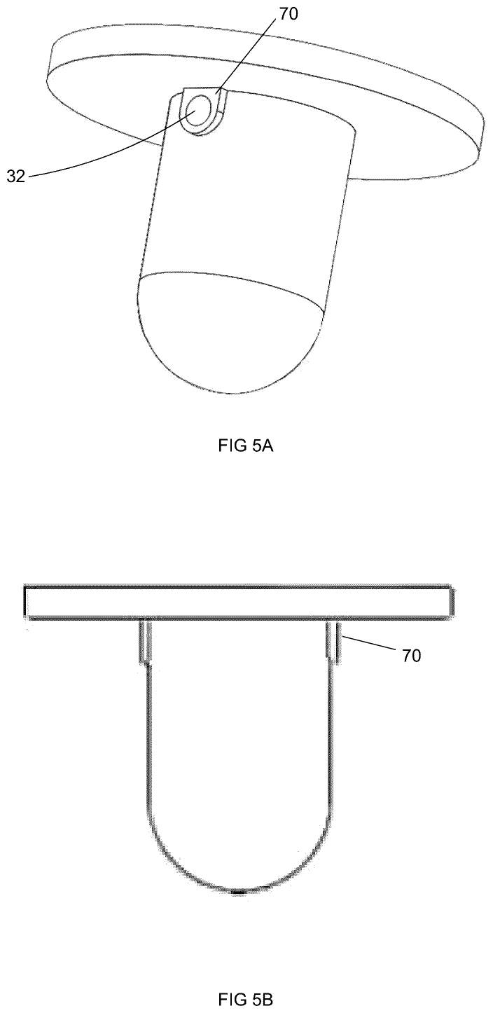

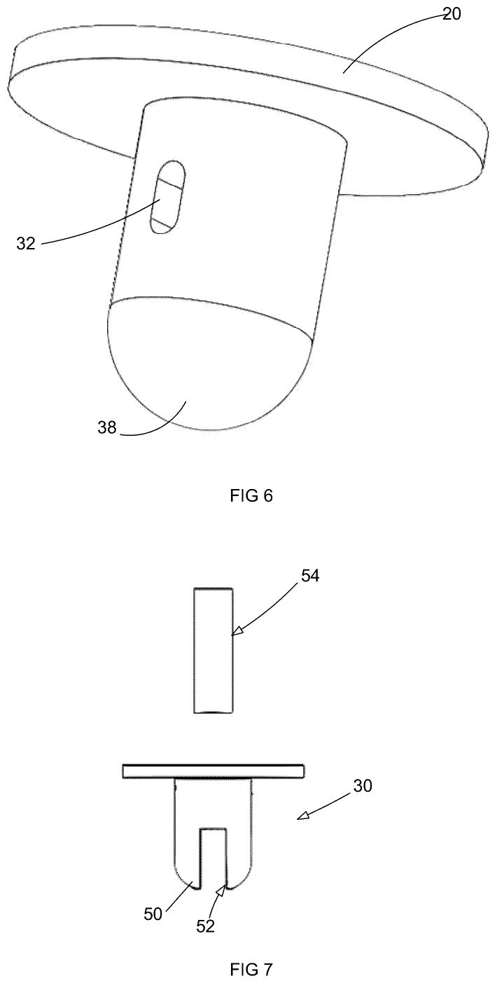

As shown by FIGS. 5A and 5B, there may be provided a protrusion wall located around an opening and exit of the through hole. This may assist with compression after insertion of the implant shaft 30 into bone.

The diameter 34 of the shaft 30 should be as small as possible in order to prevent the formation of too large of a receiving opening in the bone, but also of a diameter sufficient to receive and support the suture material without snapping or breaking. In a specific example, it has been found particularly useful to design a shaft diameter 34 that is between about 2.5-4.0 mm. In another example, the shaft diameter 34 may be between about 2.8-3.5 mm. In a specific example, the shaft diameter 34 is about 3.04 mm. The specificity of this size can be useful in identifying a properly-shaped drill bit, as described further below. The length 36 of the shaft should be as small as possible in order to prevent too deep of a receiving opening, but also deep enough that allows the implant to receive and support the suture material without pulling out of the opening. In a specific example, it has been found particularly useful to design a shaft length 36 that is between about 4.0-6.0 mm. In another example, the shaft length 34 may be between about 4.8-5.8 mm. In a specific example, the shaft length 36 is about 5.5 mm. The shaft 30 of the implant 10 may be smooth. In an alternate option, the shaft 30 may be threaded so that the implant can be twisted into place and threads can grasp internal sides of the opening prepared in the bone.

The shaft 30 extends downwardly from an implant head 20. As shown, the implant head 20 may have a circular circumference 22. As shown by FIGS. 1-3, the implant head 20 may be generally dome-shaped. For example, an upper surface 26 of the implant head 20 is formed as having a slight curvature or dome, in order for the upper surface 26 to align with patient's bone (e.g., of a curved skull) that extends around and surrounds the opening that is formed to receive the implant 10. Head edges 27 around the circumference 22 may have a slight downward taper. A lower surface 28 of the implant head 20 is shown as being formed as a generally flat lower surface 28. This can allow the lower surface 28 to directly abut with patient bone in use. It should be understood that it is also possible to provide a slightly inwardly/concave curved lower surface in order to abut with a curved skull.

In an alternate as shown by FIGS. 4-6, the implant head does not have an upper surface 26 that is curved or dome-shaped. Head edges 27 may be flat and generally perpendicular to the upper surface. Alternatively, head edges 27 may be provided with a slight downward taper so that they meet the patient's bone at a smooth transition.

In one example, a distance “D” between the upper surface 26 and the lower surface 28 (e.g., the thickness of the head 20) may be about 0.15-0.35 mm. In a more specific example, the distance “D” is about 0.25 mm. It is generally desirable for the thickness of the head to be as thin as possible so that it is not palpable once implanted, but to remain in place when the suture is pulled taut.

In one example, the implant head 20 has a diameter 24 of between about 6.0 mm-8.0 mm. In a more specific example, the implant head diameter 24 is about 7.0 mm.

Extending upwardly from the implant head 20 may be a positioner pin 40. In use, the positioner pin 40 can be used to grasp and maneuver the implant 10 into place. It is generally envisioned that once the implant 10 is positioned within the opening in bone, the positioner pin 40 can be broken off, cleanly leaving the implant head 20. In one example, tilting the positioner pin 40 away from its upward longitudinal direction (e.g., tilting it to the side) can cause the base 42 of the positioner pin 40 to break away from the implant head 20. In another example, it is possible to provide a line of weakness or a perforation option that allows the positioner pin 40 easily break away from the implant head 20. In one example, the positioner pin 40 extends up from the upper surface 26 of the implant head about 3.0-5.0 mm. In a more specific example, the positioner pin 40 extends up about 4.0 mm. The diameter 44 of the positioner pin 40 is generally envisioned as being smaller than the diameter 34 of the shaft 30. In a specific example, the diameter 44 may be about 0.5-2.0 mm. In a more specific example, the diameter may be about 1.65 mm.

Once the proper location for the tissue suspension implant 10 has been identified, the surgeon may tie off the suture through the through hole 32 and position the implant 10 within a pre-prepared opening in the bone. In some options, the shaft is a single shaft 30, and the diameter of the bone opening may be drilled slightly smaller than the diameter 34 of the shaft 30, in order to allow for a secure friction fit. The general goal is for the implant to fit snugly in the opening so that when the surgeon applies pressure on the suture to pull the eyebrow, the implant 10 will not be pulled out of the prepared opening. Exemplary drill bits and methods are described in more detail further below.

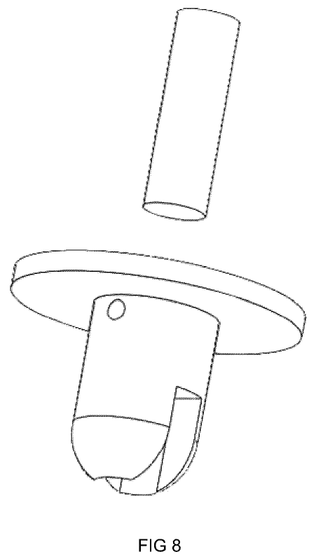

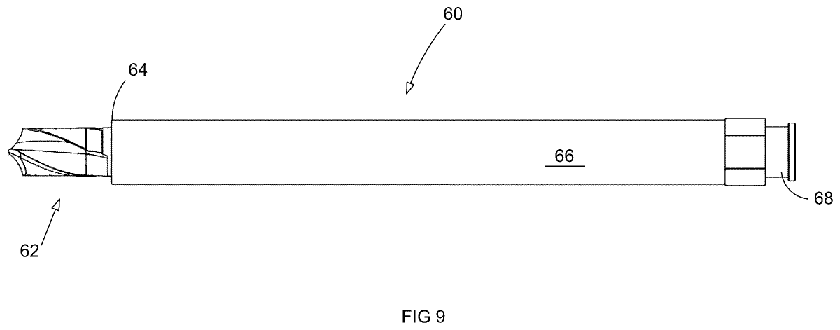

The shaft 30 may generally have a lower portion 38 that is rounded, as shown by FIGS. 4-6. However, it is possible for the lower portion 38 to be pointed (more like a pushpin), as shown by FIG. 1. It is also possible for the lower portion 38 to be flat or to have any other appropriate configuration. In an alternate example, the lower portion 38 of the shaft may be forked. This example is illustrated by FIGS. 7-8. As shown, the shaft 30 may be a split shaft with two side legs 50, 52. During implantation, the two side legs 50, 52 may be pushed slightly together in order to help secure the shaft 30 in the opening prepared in the bone. In this example, the opening prepared in bone may be almost equal to the diameter of the shaft. In this embodiment, the head of the device may be provided with a pilot hole which can receive an expander pin 54. Once the proper location of the implant has been identified, the shaft may be positioned within the pre-prepared opening in the bone, and once positioned, the expander pin 54 may be inserted into the pilot hole, in order to force the two side legs 50, 52 of the split shaft slightly apart. This can help anchor/secure the implant in place. The expander pin 54 may stay in place. The pilot hole can be deep enough or the pin can be short enough to ensure that it does not extend above the implant head. Alternatively, the expander pin 54 may be broken off similar to how the positioned pin 40 may be broken off. Various shaft shape options are possible and multiple varied implants 10 can be provided in one implant kit.

In any of the above examples, the implant 10 may also be provided with an appropriately sized drill bit 60. As shown by FIG. 9, the drill bit 60 generally has a drill bit end 62 that is sized to form an opening in a patient bone that is fractions of a millimeter smaller than the diameter 34 of the implant shaft 30. The general goal is to prepare an opening in the bone that is about 0.01 to about 0.10 millimeters smaller than the implant shaft diameter. In a specific example, the opening in the bone is drilled to be about 0.02 to about 0.05 mm smaller than the shaft diameter. In an even more specific example, the opening in bone is drilled to be about 0.04 mm smaller than the shaft diameter. For example, if the shaft 30 is designed to have a diameter of about 3.04 mm, the drill bit end 62 is designed to prepare an opening in bone that is 3.0 mm. This allows the implant shaft 30 to be press fit into the opening prepared in bone in a way that the implant 10 is not easily pulled out when pressure is applied to the suture positioned within the through hole 32.

Rearward of the drill bit end 62 is a drill bit shoulder 64. The shoulder 64 acts as a stop to prevent an opening from being prepared to deep into the bone. In one specific example, the distance between the drill bit end 62 and the shoulder 64 may be about 5-7 mm. In an even more specific example, the distance may be about 6 mm. In an even more specific example, the distance may be about 5.92 mm. Extending rearward of the drill bit shoulder 64 is the drill bit shaft 66 and a drill connection 68.

Referring now back to the implant, various materials may be used for the disclosed implant. In one specific embodiment, the implant is made of porous polyethylene. In one implementation the implant is entirely made of a biocompatible material that is composed of high-density polyethylene microspheres that are sintered to create a matrix of interconnected pores. The porosity of the material allows for rapid fibrovascular and soft tissue ingrowth and eventual incorporation of bone tissue, providing benefits of strengthening the anchoring of the implant, the anchoring of the suture attached to the implant, and decreasing the risk of infection. Polyethylene is biologically inert and has been found to trigger minimal foreign agent response in vivo, and is non-resorbable such that it will remain stable for years after implantation.

In other embodiments, the implant is made of another type of biologically inert polymer. In other embodiments the implant is made of polytetrafluoroethylene (PTFE), hydroxyappetite, polypropylene, Nylon, PEEK, PEAK, UHMW, titanium, stainless steel, or any combination thereof. For example, the shaft may be made of a different material than the head. Additionally or alternatively, one or more of the components may be made of a first material and coated with a second material.

In some implementations the implant may be impregnated prior to implantation with progenitor cells that can be selected to differentiate into a specific cell type after implantation, depending on the particular use and implantation site intended for the implant.

In some implementations the implant may be a porous, non-resorbable, and biologically inert material. The porous material may be formed by sintered particles (e.g. sintered polymer beads) such that the sintered material will define a matrix of interconnected pores. In some implementations the pores may have diameters in the range of 50 μm to 500 μm. In some implementations the pores may have diameters in the range of 100 μm to 300 μm.

The matrix of interconnected pores is configured to facilitate tissue ingrowth after implantation. After implantation, bone tissue such as cancellous and/or cortical bone tissue may grow into the interconnected pores of the implant shaft. After implantation, cortical bone tissue may grow into the interconnected pores of the lower surface of the implant head at the margins of the drilled hole into which the implant is inserted. After implantation, periosteum tissue (e.g. pericranium tissue when the implant is implanted in the cranium) may grow into the interconnected pores of the upper surface of the implant head. Due to its tissue-integrating properties, the implant will essentially become a living graft that will last the patient's lifetime. The biologically inert material will not provoke a foreign body reaction.

Additionally or alternatively, the disclosed implant may be made out of resorbable material. Non-limiting examples include a resorbable polymer, hydrogel, or any combination thereof.

Additionally or alternatively, the disclosed implant may be made out of a material that compresses when the implant is inserted into the smaller drilled opening in bone, squeezing the shaft diameter 34 with respect to the perpendicular-oriented through hole 32 with suture material inserted through. The compression of the material along the walls of the through hole 32 can add in holding the suture secure. The compression of the material also facilitates retaining the implant in the bone hole and resisting pull out, without requiring the addition of threads, ribs, or other similar structures on the smooth shaft of the implant.

The patent applicant has confirmed through testing that the disclosed implant can withstand surprising levels of pull-out resistance. Pull-to-failure testing showed that the suture (for testing a 5-0 braided polyester suture was used) would consistently break before the implant failed.

In some implementations the disclosed implant may be made out of a polymer or other material with physical properties similar to a cork material, as opposed to a relatively non-compressible material such as a as metal. A comparison between a cork versus a non-compressible, non-porous material is helpful for understanding the properties of the disclosed implant.

### Cork Material

1. **Compressibility**: Cork is naturally compressible, allowing it to deform and fit tightly within the bottle neck. This compressibility enables cork to create a tight seal even if there are minor irregularities or variations in the bottle neck's dimensions.
2. **Elasticity**: Cork has a degree of elasticity, meaning it can return to its original shape after being compressed. This property helps maintain a consistent seal over time as the cork can adapt to pressure changes within the bottle.
3. **Friction**: Due to its texture and structure, cork provides a high coefficient of friction against glass, enhancing its ability to stay in place and maintain the seal.
4. **Porosity**: Cork is porous, which allows it to compress and expand while providing a natural sealing capability. This porosity also contributes to its ability to adapt to slight variations in the bottle neck.
5. **Surface Contact**: When inserted, cork expands to fill the space within the bottle neck, creating a large contact area that maximizes the frictional force holding it in place.

### Interference Fit of Non-Compressible Material

1. **Non-Compressibility**: Materials like metals used in interference fits are generally non-compressible, meaning they do not deform easily under pressure. The fit relies on precise machining to achieve the desired tightness.
2. **Dimensional Precision**: An interference fit with non-compressible materials requires extremely tight tolerances. Both the part and the hole must be machined to precise dimensions to ensure a proper fit.
3. **Friction and Force**: The holding force in an interference fit comes from the material being forced into a slightly smaller space, generating high frictional forces. The lack of compressibility means that the parts must be pressed or shrunk fit together, often requiring significant force.
4. **Permanent Fit**: Interference fits are often more permanent compared to cork. Once fitted, removing the parts without damaging them can be challenging due to the lack of compressibility and the high frictional forces involved.
5. **Surface Contact**: The contact area in an interference fit is determined by the precision of the machining. The fit must be tight enough to prevent movement, which relies on the precise matching of the surfaces rather than the expansion and adaptability seen with cork.

In summary, cork's compressibility, elasticity, and porosity make it ideal for sealing applications where minor irregularities exist and a flexible, adaptable fit is beneficial. In contrast, an interference fit with non-compressible materials relies on precise machining and high frictional forces to achieve a tight, often permanent fit.

The disclosed implants, in at least some implementations, may have similar compressibility, elasticity, friction, porosity, and surface contact properties as cork. For example, in some implementations, the disclosed implants may be made with a relatively compressible material having a Young's modulus on the order of megapascals (e.g. less than 1,000,000 pascals) as opposed to materials such as many metals, which have a Young's modulus on the order of gigapascals (e.g. up to 999,000,000,000 pascals). As another example, in these or other implementations, the disclosed implants may be made with a relatively elastic material, allowing it to be compressed to up to 95%, up to 90%, or up to 80% of its original dimension without significant permanent deformation.

The dimensions and material examples provided herein are provided for exemplary purposes only. It is envisioned that a single implant size may be provided in order to ease inventory, but it should also be understood that alternate sizes may be designed and provided.

One exemplary procedure for using the device described herein as outlined below. It should be understood that surgical techniques may vary by practitioner; these steps are intended to be illustrative only and not limiting in any way. FIGS. 10 shows an exemplary views of implantation surgery once preparation of the implant site has been completed. FIG. 11 shows a view of the implant site once the tissue suspension implant has been positioned.
Step 1: Drill opening into bone location determined by the surgeon
Step 2: Thread a suture through the implant through hole. The suture may be secured to the implant using a knot similar to a sailors knot.
Step 3: Position the implant shaft into the opening perpendicular to the bone and push straight down.
Step 4: Pass the suture through periosteal tissue and adjust suspension of tissue to where desired.

The subject matter of certain embodiments of this disclosure is described with specificity to meet statutory requirements, but this description is not necessarily intended to limit the scope of the claims. The claimed subject matter may be embodied in other ways, may include different elements or steps, and may be used in conjunction with other existing or future technologies. This description should not be interpreted as implying any particular order or arrangement among or between various steps or elements except when the order of individual steps or arrangement of elements is explicitly described.

It should be understood that different arrangements of the components depicted in the drawings or described above, as well as components and steps not shown or described are possible. Similarly, some features and sub-combinations are useful and may be employed without reference to other features and sub-combinations. Embodiments of the invention have been described for illustrative and not restrictive purposes, and alternative embodiments will become apparent to readers of this patent. Accordingly, the present invention is not limited to the embodiments described above or depicted in the drawings, and various embodiments and modifications may be made without departing from the scope of the claims below.

What is claimed is:

1. A bone anchor comprising:
(a) an implant head comprising a lower surface and an upper surface, wherein the lower surface is flat or concave;
(b) an implant shaft extending down from the lower surface of the implant head;
(c) a through hole extending laterally through the implant shaft below the implant head; and
(d) wherein the bone anchor is made of a porous material.

2. The bone anchor of claim 1, wherein the upper surface of the implant head is domed or flat.

3. The bone anchor of claim 2, wherein the implant head is domed and tapers toward an outer edge of the implant head.

4. The bone anchor of claim 2, wherein the implant head comprises a maximum thickness that is 0.35 mm or less.

5. The bone anchor of claim 4, wherein the implant head comprises an outer diameter and the implant shaft comprises an outer diameter, wherein the outer diameter of the implant head is at least 2 mm wider than the outer diameter of the implant shaft.

6. The bone anchor of claim 5, wherein the implant shaft is smooth.

7. The bone anchor of claim 6, wherein the porous material is a porous polyethylene.

8. The bone anchor of claim 1, further comprising a positioner pin extending upward from the upper surface of the implant head for use in positioning the bone anchor, the positioner pin configured to be broken off from the implant head.

9. The bone anchor of claim 1, wherein the bone anchor is a tissue suspension implant for anchoring a suture in a bone.

10. The bone anchor of claim 1, wherein the porous material is a non-resorbable material.

11. The bone anchor of claim 10, wherein the porous material is formed of sintered particles defining a matrix of interconnected pores.

12. The bone anchor of claim 11, wherein the porous material is a biologically inert polymer.

13. The bone anchor of claim 11, wherein the pores have diameters in the range of 50 μm to 500 μm.

14. The bone anchor of claim 11, wherein the pores have diameters in the range of 100 μm to 300 μm.

15. The bone anchor of claim 11, wherein the pores are tissue ingrowth pores.

16. The bone anchor of claim 11, wherein the porous material is polyethylene, polytetrafluoroethylene, Nylon, hydroxyappetite, polypropylene, PEEK, PEAK, UHMW, titanium, stainless steel, or any combination thereof.

17. A bone anchoring method comprising:
(a) surgically exposing a bone tissue beneath a periosteum tissue;
(b) forming a hole in the bone tissue;
(c) inserting an implant shaft of a bone anchor into the hole formed in the bone tissue such that the shaft compresses as it is inserted into the hole to retain the bone anchor in the hole, wherein the bone anchor comprises:
(i) an implant head comprising a lower surface and an upper surface, wherein the lower surface is flat or concave;
(ii) the implant shaft extending down from the lower surface of the implant head;
(iii) a through hole extending laterally through the implant shaft below the implant head; and
(iv) wherein the bone anchor is made of a porous material.

18. The bone anchoring method of claim 17, wherein, after inserting the implant shaft of the bone anchor into the hole, the implant shaft is in contact with cancellous bone tissue, the lower surface of the implant head is in contact with cortical bone tissue, and the upper surface of the implant head is in contact with the periosteum tissue.

19. The bone anchoring method of claim 18, wherein, after inserting the implant shaft of the bone anchor into the hole, pores of the implant shaft allow ingrowth of the cancellous bone tissue and pores of the upper surface of the implant head allow ingrowth of the periosteum tissue.

20. The bone anchoring method of claim 17, further comprising:
(a) wherein a suture extends through the through hole of the bone anchor prior to insertion of the shaft of the bone anchor into the hole; and
(b) after inserting the implant shaft of the bone anchor into the hole, tensioning the suture.

21. The bone anchoring method of claim 17, wherein tensioning the suture comprises passing the suture through a portion of the periosteum tissue and adjusting suspension of the portion of the periosteum tissue.

22. The bone anchoring method of claim 21, wherein the bone tissue is cranial tissue.

23. The bone anchoring method of claim 17, wherein a positioner pin extending upwardly from the implant head is used to insert the shaft of the bone anchor into the hole; and, wherein, after inserting the shaft of the bone anchor into the hole, the positioner pin is broken off from the implant head.

24. The bone anchoring method of claim 17, wherein forming the hole in the bone tissue comprises forming a hole of a first diameter in the bone tissue, wherein the implant shaft comprises a second diameter, wherein the first diameter is smaller than the second diameter.

25. The bone anchoring method of claim 17, wherein the porous material is a non-resorbable material.

26. The bone anchoring method of claim 17, further comprising, prior to implantation. impregnating the bone anchor with progenitor cells.
